(19) United States
(12) Patent Application Publication
(10) Pub. No.: US 2022/0151789 A1
(43) Pub. Date: May 19, 2022

(54) METHOD FOR ADJUSTING MECHANICAL PROPERTIES OF IMPLANT AND PATIENT SPECIFIC SURGICAL IMPLANTS

(71) Applicant: GEORGIA TECH RESEARCH CORPORATION, Atlanta, GA (US)

(72) Inventors: Seung-Kyum Choi, Marietta, GA (US); Mahmoud Alzahrani, Atlanta, GA (US); Recep Gorguluarslan, Cankaya/Ankara (TR)

(73) Assignee: GEORGIA TECH RESEARCH CORPORATION

(21) Appl. No.: 17/069,135

(22) Filed: Oct. 13, 2020

Related U.S. Application Data
(63) Continuation-in-part of application No. 16/263,254, filed on Jan. 31, 2019, now abandoned.

(30) Foreign Application Priority Data
Aug. 14, 2018 (KR) 10-2018-0095176

(51) Int. Cl. A61F 2/30; G06F 30/10

(52) U.S. Cl. CPC A61F 2/30942; G06F 30/10; A61F 2/32; A61F 2002/30069; A61F 2002/30952; A61F 2002/30948

(57) ABSTRACT

The present invention is for a systematic process of creating patient-specific implants by matching target mechanical properties (e.g., elastic modulus of bone) based on the bone density information from a patient's CT scan images. The present invention creates lattice scaffolds using conformal unit-cells while minimizing the deviations between as-fabricated scaffolds and as-designed scaffolds. The present invention also creates a metamodel that matches the elastic modulus values of lattice scaffolds to desired values by using a homogenization approach to determine the characteristics of the lattice structure at the unit-cell level. The utilization of the metamodel enables designing the scaffolds without requiring any optimization procedure.

### REFERENCE TO RELATED APPLICATIONS

This is a continuation in part of pending U.S. patent application Ser. No. 16/263,254 filed on Jan. 31, 2019, which claims the priority benefit of Korean Patent Application No. 10-2018-0095176 filed on Aug. 14, 2018, which claims the benefit of U.S. Provisional Patent Application No. 62/626,749, filed on Feb. 6, 2018 in the U.S. Patent and Trademark Office, the entire contents of which are incorporated herein by reference.

### FIELD OF THE INVENTION

The disclosure relates to a method which adjusts the mechanical properties of implants that have a lattice scaffold structure and patient specific surgical implants created according to the method.

### BACKGROUND OF THE INVENTION

The term, implant, is widely used to mean any medical device manufactured to replace or improve biological structures, including artificial organs/joints and dental implants. It is possible to surgically integrate artificially made implants into a patient's bones or tissues. In tissue engineering, which involves artificially made biological tissues for surgical purposes, lattice scaffolds, often used in the field of construction, are introduced to improve or replace biological tissues in the design of various implants.

In general, implants such as artificial joints are usually made of metals and polymers. When a metal-based implant (e.g. stainless steel or titanium-based implant) is inserted into a bone of a human body through surgical operations, the patient might need reoperations several years later. This is necessary because repeated use of the implant generates stresses around the lesion area of the implant and results in bone deformation and even cracks. The largest cause behind this side effect is that the difference in mechanical properties, such as elastic modulus, between the bone and the implant material is quite large.

This issue is called “Stress Shielding” in tissue engineering. Because of the stress shielding, it is quite common for patients to consider reoperations after a certain period of time in order to handle bone damages and loosened implants.

### SUMMARY OF THE INVENTION

An aspect of the disclosure is to provide surgical implants that match the mechanical properties (i.e. elastic modulus) of the patient's bone tissues by manipulating the shape and dimensions of the lattice scaffolds. This aspect can be achieved by adjusting the shape, size, and dimension of the unit-cell in the lattice structures of the implant. By utilizing the lattice scaffolds which have similar mechanical properties to the patient's bone, patient specific implants can be produced for artificial joints, bone tissues, teeth, etc. such that the implant can be used for a long period of time without worrying about reoperations.

Another aspect of the disclosure is to provide surgical implants that have various elastic modulus values according to the identified elastic modulus obtained from CT scan images such that the patient specific implant has optimal elastic modulus values over its domain. With this configuration, it is provided to minimize the stress shielding effect.

According to an embodiment of the disclosure, there is provided a method for adjusting mechanical properties of an implant. The method includes: specifying a region requiring an implant operation from a CT scan image of a patient's affected part, determining an implant shape to be inserted into the specified region, dividing the implant shape into a plurality of partitioned three-dimensional regions, assigning a target elastic modulus value (Et) for each of the plurality of partitioned three-dimensional regions, selecting one of a many types of lattice scaffolds for the implant, selecting a material for the implant, and adjusting a strut diameter and/or a density of the selected type of the lattice scaffolds to minimize the difference between the target elastic modulus (Et) and a homogenized elastic modulus (Eh), which is calculated based on the implant material's elastic modulus value (Eo), and information obtained from the partitioned three-dimensional regions.

The plurality of partitioned three-dimensional regions comprises a plurality of conformal unit-cells with a matching target elastic modulus value from a tensor containing coordinates and bone density information.

The plurality of partitioned three-dimensional conformal unit cells may include neighboring regions where the target elastic modulus (Et) are different, thus resulting in those regions being connected by unit cells which have different strut diameters and/or densities.

The method may further include: creating a plurality of three-dimensional voxel meshes based on the partitioned three-dimensional region; adjusting the size of the three-dimensional region to correspond to the manufacturing requirements of the lattice scaffolds; and assigning a target elastic modulus value to each of the plurality of three-dimensional conformal unit cells generated from the resized voxel mesh according to the bone density information obtained from the CT scan images.

The size of the three-dimensional conformal unit-cells is adjusted while minimizing density information to satisfy other practical requirements, such as minimum print features, and while minimizing the effect on matching Et, by using a modified Structural Similarity Index Method (SSIM) that is developed to handle three dimensional tensors.

The homogenized elastic modulus (Eh) for a unit-cell of the as-printed lattice scaffolds may be estimated by using a multiscale modeling method based on the elastic modulus of the implant material (Eo) that account for the variations between the as-designed and as-printed scaffolds.

Design parameters of unit-cells of the lattice scaffolds may be determined by using a metamodel that utilizes a unit-cell size (L), a normalized elastic modulus (Eh/Eo), and a unit-cell density (ρ) without any optimization process.

Any type of conformal unit-cell is selected from several types, including, but not limited to, crossed, cantley, octet, Paramount1, Diagonal, Paramount2, Midpoint, or tetrahedral or body centered cubic (BCC) unit cell.

The patient specific surgical implant, which has precisely designed lattice scaffolds according to the adjustment method of the present invention, can be used for long periods of time because the stress shielding effect can be reduced by matching the elastic modulus of the as-printed implant to the elastic modulus of the bone of the affected area. Therefore, the present invention can significantly reduce the side effect of the implant and prevent reoperations.

### BRIEF DESCRIPTION OF THE DRAWINGS

The above and/or other aspects will become apparent and more readily appreciated from the following description of exemplary embodiments, taken in conjunction with the accompanying drawings, in which:

FIG. 1A is a flowchart showing a process of making a conformal patient specific implant according to an exemplary embodiment of the disclosure; FIG. 1B shows the main difference between conformal and non-conformal lattice structures.

FIGS. 2A and 2B are conceptual diagrams showing a process of forming a conformal patient specific implant according to an exemplary embodiment of the disclosure;

FIG. 3 is a flowchart showing an algorithm according to generate the patient specific implant by using conformal lattice structures with predefined unit-cells and metamodels according to the exemplary embodiment of the disclosure;

FIG. 4 is a flowchart showing an alternative algorithm to generate the patient specific implant by using optimization methods according to the exemplary embodiment of the disclosure;

FIGS. 5A to 5G show examples of the unit-cell types within the three-dimensional voxel mesh to generate conformal lattice scaffolds for a patient specific implant according to the exemplary embodiment of the disclosure;

FIGS. 6A and 6B show a patient specific implant which can be applied to femur surgery according to the exemplary embodiment of the disclosure;

FIGS. 7A and 7B show a patient specific lumbar interbody fusion implant which can be applied to spinal fusion surgery according to the exemplary embodiment of the disclosure;

FIGS. 8A and 8B show a patient specific hip replacement implant which can be applied to hip joint surgery according to the exemplary embodiment of the disclosure;

FIGS. 9A to 9D show graphical representations of the relationships between design parameters of the implant according to the exemplary embodiment of the disclosure: a normalized elastic modulus value of the metamodel and corresponding contour plot according to the size and density of the as-printed unit-cell.

FIGS. 10A and 10B show the as-designed strut members of unit cells with constant diameter and material properties; and the as-fabricated struts with variations in diameter and heterogeneous material with porosity included (dark areas), which will influence the resulting homogenized properties of the unit cells, depending on the additive manufacturing technique.

### DETAILED DESCRIPTION OF THE INVENTION

Below, embodiments of the disclosure will be described in detail with reference to accompanying drawings so it can be easily actualized by a person having an ordinary skill in the art. The disclosure may be achieved in various different forms without being limited to the embodiments set forth herein. For clarity of description, like numerals refer to like elements throughout.

As the average life span of human beings has been extended due to the rapid developments of scientific technologies, extensive research work has been done on the development of various implants, including artificial bones, joints, and teeth to treat critical damages of the human body generated from accidents, cancer, stress, obesity, etc. The implants should be non-toxic and be able to maintain their original function in harmony with the living tissues of the affected area. Although significant efforts have been made to develop superior biomaterials for these implants, the elastic modulus of the existing implant materials is considerably different (about 3 to 10 times different) from the elastic modulus of the bone. Due to this significant difference, the implant induces stress shielding in vivo after 2 to 4 years of living with the implant. Thus, considering a reoperation becomes inevitable.

When the difference in elastic modulus between bone tissues and implants is large, the implant (usually made of material with a high elastic modulus value) supports most of the stresses/loads transmitted to the bone. If the living tissues of the bone are not subject to tensile, compressive, and bending stresses for long term, the human body automatically recognizes that it does not need the role of the bone in that specific area. Thus, the bone thickness and weight are automatically reduced which causes osteoporosis around the implant. As a result, the cohesion between the implant and the bone near the insertion point becomes weak and causes the stress shielding phenomenon. Therefore, when stress shielding occurs, the bone density around the implant is reduced and the implant is easily separated from the implantation site over time. In addition, when the implants are made of metals, the implants sometimes damage bone tissues in the surrounding area since its strength is considerably larger than the bone.

Therefore, the disclosure provides to reduce the stress shielding effect of the implant while still making the implant from common metal materials available in manufacturing. In these days, tantalum, palladium, zirconium, niobium and titanium alloys (i.e., Ti-6AI-4V) are commonly used for implants. In general, the elastic modulus of the bone is around 5 GPa, although sometimes it can be as high as 10 to 40 GPa near areas that suffer from high loads/stresses. Thus, the elastic modulus value of the implant material (e.g., the elastic modulus value of Ti alloys is 110 GPa) is about 3 to 10 times greater compared to the bone's elastic modulus value.

The disclosure suggests an adjustment method for matching the elastic modulus of the implant to the elastic modulus of bone tissues regardless of the implant materials. It can be applied not only to existing implant materials but also to future implant materials. Computationally, the disclosure can use a designed lattice scaffold to find a matched elastic modulus value of the as-printed one without an optimization, as long as the elastic modulus of the material is given. The following descriptions provide the detailed explanations of the disclosure along with accompanying drawings.

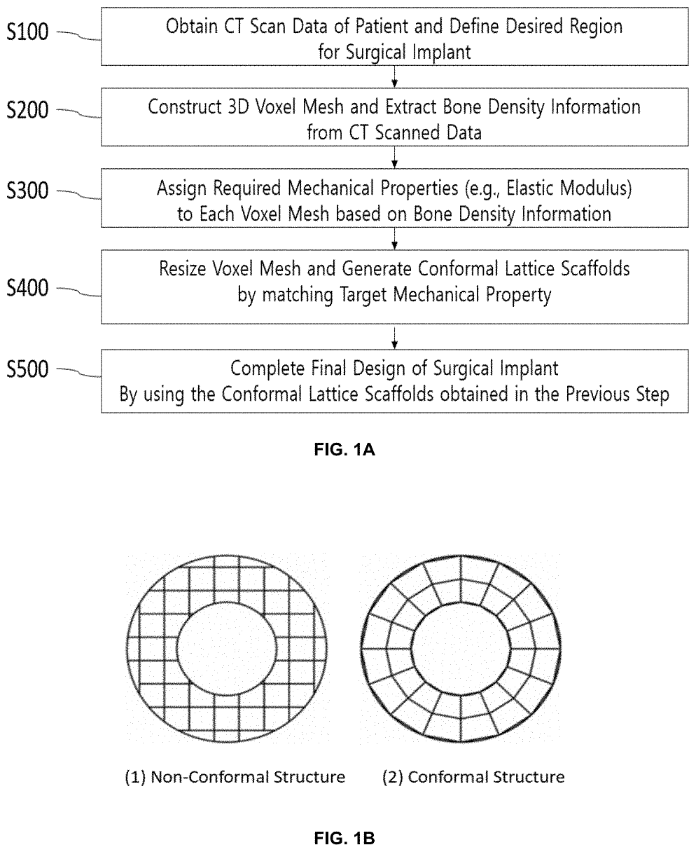

FIG. 1A is a flowchart showing a process of making a patient-specific implant according to the exemplary embodiment of the disclosure. It is provided with five operations including 1) defining the desired region for the implant from the CT scan data of a patient (S100), 2) creating the three-dimensional voxel mesh and extracting bone density information from the CT scan data (S200), 3) assigning a target mechanical property such as elastic modulus value for each voxel mesh (S300), 4) resizing the voxel mesh and generating lattice scaffolds (S400), and 5) producing the surgical implant with the lattice scaffolds (S500).

The exemplary embodiment first obtains CT scan images of the lesion of a patient and specifies the region requiring the implantation at operation S100. The exemplary embodiment can make the patient specific implant by using the obtained CT scan images on the affected area of the patient.

In a second operation, the bone density information for each voxel mesh is extracted from the three-dimensional bone model (S200). Although the CT scan images are two-dimensional images, it is possible to construct a three-dimensional bone model for the lesion portion by stacking two-dimensional CT scan images. By using the three-dimensional bone model based on the CT scan images, both the shape of the implant to be inserted and the inserted shape of the bone's lesion portion can be determined. Then, the three-dimensional voxel mesh is generated according to the three-dimensional bone model which contains the detailed information from the CT scan data. Also, the bone density information for each position distributed over the bone's lesion to be inserted is extracted from the three-dimensional bone model which is constructed from two-dimensional CT scan images. The unit three-dimensional voxel mesh is a basis of the implant shape and it also represents a unit divided region corresponding to each position obtained by dividing the three-dimensional bone model into multiple partitioned three-dimensional regions. When the specific type of unit-cell is selected, the selected unit-cells are allocated to each voxel mesh. Then, the voxel mesh filled with the unit-cells becomes the lattice scaffolds which are utilized to form the patient specific implant. The mechanical properties or the elastic modulus values of the implant are adjusted for each unit-cell of the lattice scaffolds in the following operations.

In a third operation, the target elastic modulus values are assigned for each voxel mesh based on the extracted bone density information (S300). The target elastic modulus value can be calculated based on the bone density information since it is known that their relationship is highly correlated. The relationship and analytic formula between the bone density and bone's mechanical properties are available in existing literatures with reference to Reference documents 9, 10 listed in U.S. provisional application No. 62/626,749.

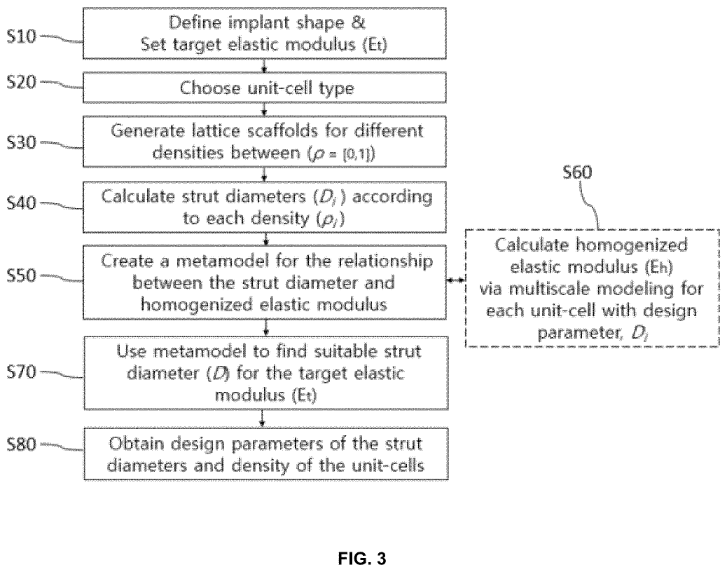

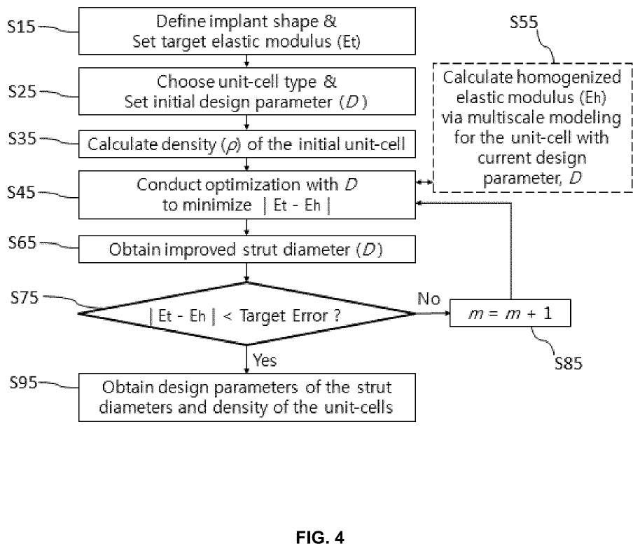

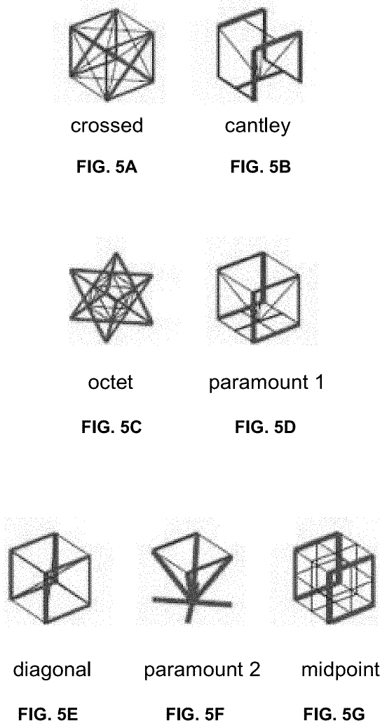

In a fourth operation, the size of the voxel mesh is adjusted and the type of the unit-cell for the lattice scaffold is selected. Then, the initial lattice scaffolds using the selected unit-cells are generated within the three-dimensional voxel mesh at operation S400. The scaffolds are often used in building constructions and their shape is usually formed as lattice structures. A similar shape is utilized in the surgical implant in the disclosure and it is called the lattice scaffold which is formed with unit-cell structures. There are many different types of unit-cell structures (FIG. 5). One specific type of unit-cell is selected, and that becomes the basis to form the lattice scaffolds for the implant. Within the unit-cell structure, the diameter of each strut can be adjusted to match the elastic modulus of the printed lattice scaffold to the target elastic modulus of the bone. The adjustment methods for the strut diameter and density of the unit-cell are described in FIGS. 3 and 4. In the disclosure, any types of the unit-cell structures can be utilized to form the lattice scaffolds.

In a fifth operation, the lattice scaffolds generated from another operation are applied to form the patient specific implant. Then, the implant is fabricated using a 3D printer at operation S500. The disclosure produces a patient specific implant which has a similar elastic modulus to that of the patient's bone, so that the effect of stress shielding can be reduced.

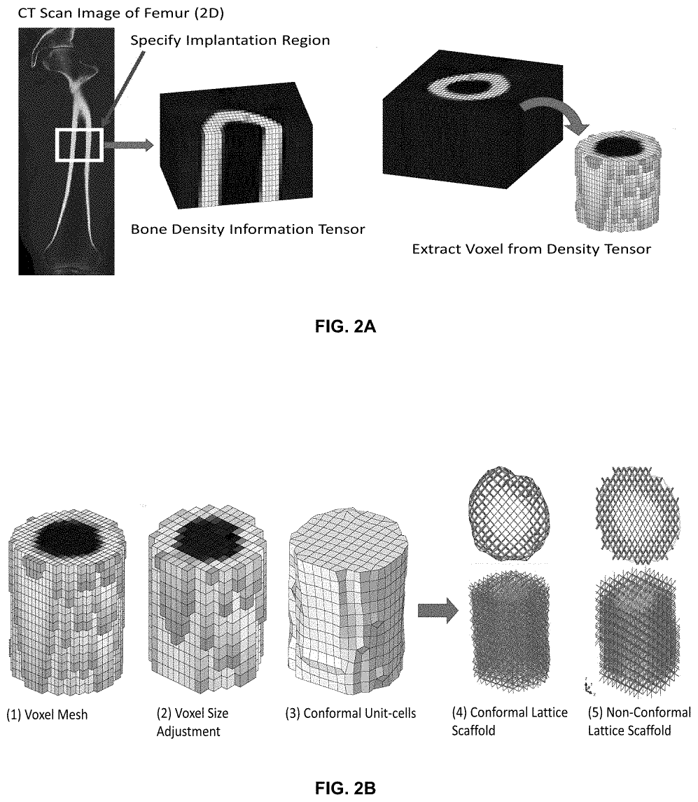

FIG. 2 is a schematic diagram showing a process of manufacturing a patient specific implant according to an exemplary embodiment of the disclosure. FIG. 2 provides additional detailed information about the operations presented in the flowchart of FIG. 1A.

FIG. 2A, summarizes a procedure of how to generate a three-dimensional tensor from two-dimensional CT scan images of a patient's femur and how the three-dimensional voxel mesh, which is going to be the basis of the implant shape, is generated based on the tensor. In addition, the bone density information is extracted from the gray scale information of the CT scan image of the corresponding region in the tensor. Each element in the tensor is colored as white or light gray where the bone density is high; otherwise, it is colored as dark gray or black where the bone density is low. The target elastic modulus value for each unit cell is determined based on the general relationship between the bone density and corresponding elastic modulus.

FIG. 2B shows the (1) voxel mesh, (2) adjusted mesh size, (3) generated conformal unit cells, (4) generated conformal lattice scaffolds, and (5) non-conformal lattice scaffold for comparison. The initial size of the voxel mesh based on the original tensor, generated based on the CT scan images, is often too small to generate unit-cells to be fabricated for the lattice scaffolds of the implant (FIG. 2B-(1)). Thus, it is necessary to adjust the size of the voxel mesh. The method presented in the disclosure is applicable to any size of tensors used to generate the voxel mesh, but it is normally adjusted as 2 mm to 6 mm size for the three-dimensional implant. In the disclosure, a three-dimensional modified Structural Similarity Index Method (SSIM) is used to minimize the loss of image data information when the size of the tensor is adjusted based on computational and manufacturing requirements (FIG. 2B-(2)). This additional effort is necessary to enclose accurate bone density information for each voxel mesh by minimizing the image information loss. After resizing the voxel mesh using the resized tensor, the basic conformal unit-cells are generated (FIG. 2B-(3)). Once the basic unit-cells are generated, one specific type of the unit-cell is selected and the entire design region will be filled with the conformal unit-cell structures. Then, finally the initial creation of the conformal lattice scaffolds is completed (FIG. 2B-(4)). A design using non-conformal unit-cells is shown for comparison in FIG. 2B-(5).

After generating the initial lattice scaffolds, the target mechanical property or target elastic modulus of the implant is achieved by using the disclosure's algorithm, which adjusts the strut's diameter or density of the unit-cell structures in the lattice scaffolds by accounting for the deviation between the diameters of the as-designed and as-printed unit-cells without the need for any optimization procedure. These strut diameters and the densities of the unit-cells are adjusted to match the target elastic modulus values by using the disclosure's algorithm (FIG. 3). As shown in FIG. 2B-(4), the diameters of the struts are varying for each unit-cell to match the target elastic modulus values. As a result, the strut diameters of the unit-cells corresponding to the regions that have relatively high bone density (e.g., white colored region) are larger than the strut diameters of the unit-cells corresponding to the voxel mesh regions that have relatively low bone density (e.g., dark gray or black region).

FIG. 3 shows a flowchart for an algorithm to adjust the elastic modulus of the lattice scaffolds by changing the strut diameters and densities of the unit-cells of the lattice scaffolds to make a patient specific implant according to the exemplary embodiment of the disclosure.

In the disclosure, the implant's shape is formed based on the multiple three-dimensional regions. The three-dimensional regions are specified by the multiple three-dimensional voxel meshes. When making a patient specific implant through the disclosure, the shape of the implant is determined from the CT scan images on the patient's affected area, and the target elastic modulus value is assigned for each voxel mesh at operation S10. The target elastic modulus (Et) for each voxel mesh can be assigned according to the bone density information from the CT scan images. However, when the CT scan images are not available, it is also possible to select most appropriate elastic modulus values from a predetermined database or existing literatures based on the patients' sex, age, and characteristics of the affected parts.

The next operation is to select a specific type of unit-cell from a unit-cell structure library (S20). An example of the unit-cell library for the various types of unit-cells is available in FIG. 5.

It is provided to generate various unit-cells by varying strut diameters according to many different densities of p at operation S30. The density of the unit-cell represents the ratio between the volume of the material utilized to form the struts of the unit-cell and the total volume of the unit-cell space. Thus, a density of 1.0 means the entire volume of the unit-cell is fully filled with solid materials. And a density of 0.0 means there are no materials in the inside of the unit-cell volume.

Then the strut diameters of D are calculated according to the density of each unit-cell at operation S40.

A metamodel is generated for the relationship between the strut diameter and the elastic modulus of the implant at operation S50. In the operation of S30, various unit-cell structures were created according to many different density values (e.g., 0.0, 0.1, 0.2, 0.3, 0.4, etc.). The effective physical properties are determined for the as-designed unit cells, which will differ when they are fabricated due to manufacturing variations. Then, those unit cells can be fabricated by the target additive manufacturing technique to identify the differences between the as-designed and as-printed diameters corresponding to the density values. In the current operation of S50, a metamodel for the relationship is created between the elastic modulus and the strut diameters of the unit-cells, which were created for many different density values by including the differences when additively manufactured.

When the elastic modulus is estimated based on the as-printed strut diameter of the unit-cell in operation S50, a multi-scale modeling method is used to obtain the homogenized elastic modulus value for the unit-cell structures at operation S60. The homogenized elastic modulus value can be calculated for each unit-cell or the entire domain of the lattice scaffolds which includes multiple unit-cells. Since the patient specific implant is formed using lattice scaffolds which are not solid over the volume, the elastic modulus of the implant is different from the elastic modulus of the implant material. Thus, the actual elastic modulus of the implant is obtained as a homogenized elastic modulus (Eh) by using a multiscale modeling method based on the implant material's elastic modulus (Eo).

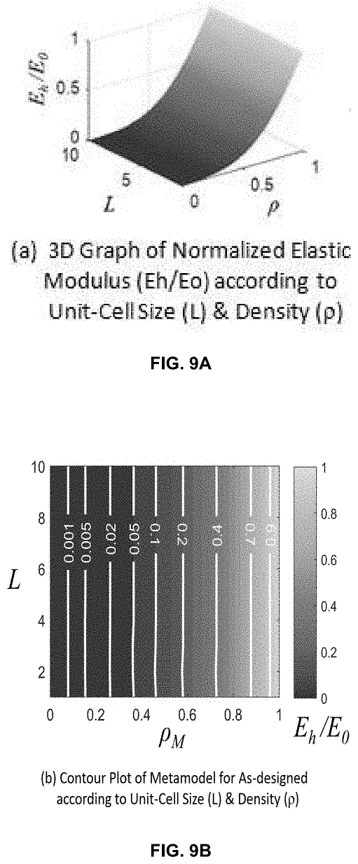

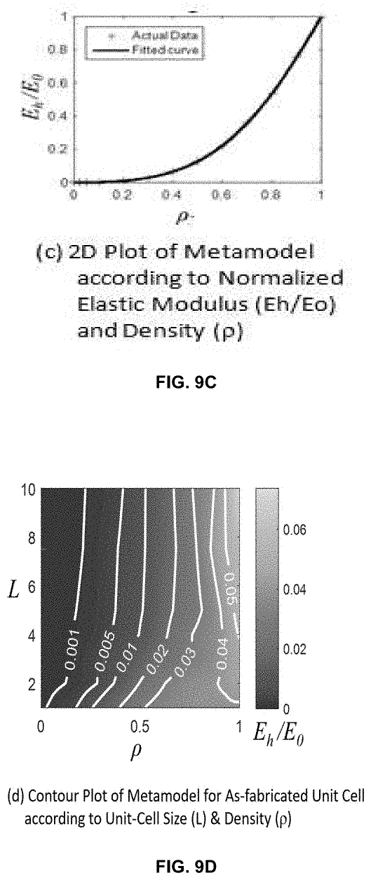

By using the metamodel, suitable as-printed strut diameters of unit-cells are determined to match the target elastic modulus without requiring any optimization procedure at operation S70. Once the metamodel (S50) is generated for the relationship between the homogenized elastic modulus values (S60), unit-cell type, unit-cell size, and strut diameter, it is possible to determine the appropriate strut diameters and densities of the unit-cells according to the target elastic modulus value by using the identified metamodel. This process does not require any additional numerical or optimization procedures. The entire design space is represented with the metamodel and suitable design parameters are directly identified from the design space. For instance, the relationship between the homogenized elastic modulus values, unit-cell densities, and strut diameters can be depicted in multi-dimensional spaces. FIG. 9 (a) and (b) illustrates the multi-dimensional graphs for the relationships between the unit-cell size (L), density (ρ), and normalized elastic modulus value (Eh/Eo). The details of FIG. 9 are discussed later.

The identified design parameters are obtained including the strut diameter, density, and size of the unit-cell at operation S80. These parameters are utilized in the fourth operation (S400) of FIG. 1.

FIG. 4 is a flowchart which describes an alternative algorithm to determine the design parameters for a patient specific implant according to the exemplary embodiment of the disclosure. Instead of using the process described in FIG. 3, similar values of the design parameters can be obtained using the process in FIG. 4.

In the process of making a patient specific implant through the exemplary embodiment, the shape of the implant is determined from the CT scan images on the patient's affected area. Also, the target elastic modulus value (Et) is assigned for each voxel mesh at operation S15. The target elastic modulus (Et) for each voxel mesh can be assigned according to the bone density information from the CT scan images. However, when the CT scan images are not available, it is also possible to select most appropriate elastic modulus values from a predetermined database or existing literatures based on patients' sex, age, and characteristics of the affected parts.

A specific type of unit-cell is selected from a unit-cell structure library at operation S25. An example of the unit-cell library for the various types of unit-cells is available in FIG. 5.

An initial density (ρ) is set for a candidate unit-cell at operation S35. The density of the unit-cell represents the ratio between the volume of the material utilized to form the struts of the unit-cell and the total volume of the unit-cell space. Thus, a density of 1.0 means the entire volume of the unit-cell is fully filled with solid materials. And a density of 0.0 means there are no materials in the inside of the unit-cell volume.

Conduct any of the conventional optimization algorithms to minimize the difference between the target elastic modulus (Et) and the homogenized elastic modulus (Eh) at operation S45. Unlike the method described in FIG. 3, a metamodel which describes the relationship between the elastic modulus and the strut diameter of the unit-cell is not created in this process.

When conducting the optimization, the homogenized elastic modulus (Eh) is estimated by using a mutli-scale modeling method based on the implant material's elastic modulus (Eo) at operation S55. This estimation process for the homogenized elastic modulus is the same as described in FIG. 3.

The strut diameter (D) of the unit-cell is obtained at operation S65. By utilizing a conventional optimization algorithm, the strut diameter, which minimizes the difference between the target elastic modulus (Et) and the homogenized elastic modulus (Eh), can be obtained.

It is checked whether the difference between the target elastic modulus and the homogenized elastic modulus is within a predefined error range at operation S75.

If the difference between the target elastic modulus and the homogenized elastic modulus is larger than the predefined error range, repeat the optimization process of operation S45 and increase the iteration number of “m” at operation S85.

When the difference between the target elastic modulus and the homogenized elastic modulus is less than the predefined error, obtain the design parameters including the strut diameter, size, and density of the unit-cells at operation S95. These parameters are utilized in the fourth operation (S400) of FIG. 1. By using the processes described in FIG. 4, the same process is repeated to determine the design parameters for many different types of the unit-cells. Although this process does not require constructing a metamodel (FIG. 9), the optimization process may induce more computational costs compared to the algorithm described in FIG. 3.

FIGS. 5A-5G show various types of the three-dimensional unit-cell structures. These unit-cell structures are the basis of forming the lattice scaffolds. Along with the examples shown in FIGS. 5A-5G, other types of the unit-cell structures can also be utilized in the disclosure. The disclosure does not have any limitations on the shape of the unit-cell structures. FIGS. 5A-5G show the examples of various unit-cell structures including ‘crossed’ (FIG. 5A), ‘cantley’ (FIG. 5B), ‘octet’ (FIG. 5C), ‘paramount 1’ (FIG. 5D), ‘diagonal’ (FIG. 5E), ‘paramount 2’ (FIG. 5F) and ‘midpoint’ (FIG. 5G). Along with these unit-cell types, other types of unit-cell structures can also be used in the disclosure. For instance, the example presented in FIG. 2 utilized a popular type of the unit-cell; namely the BCC (Body-Centered Cubic) unit-cell.

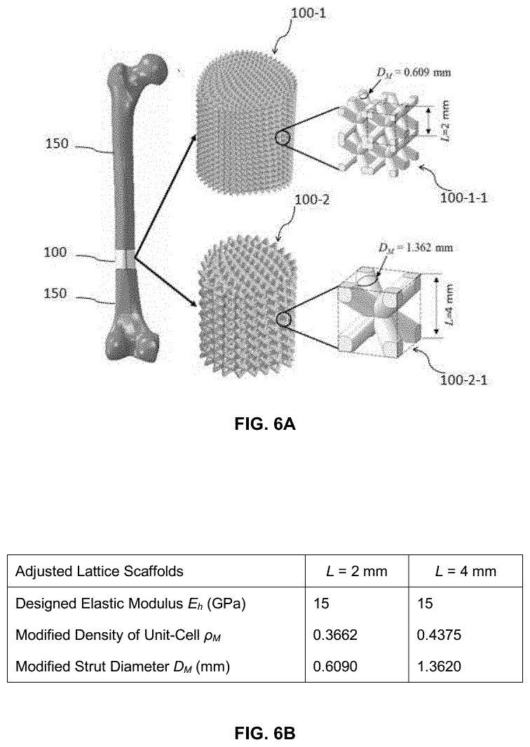

FIG. 6 shows an example of a patient specific implant for femoral bone segment according to an exemplary embodiment of the disclosure. For instance, when a patient has bone cancer in the area of femoral bone segment, the corresponding bone portion of the lesion is replaced by the artificial implant that matches the bone's elastic modulus according to the exemplary embodiment of the disclosure.

In FIG. 6A, two different patient specific implants for the femoral bone designed according to this exemplary embodiment are shown. A femoral bone implant (100) made of the lattice scaffolds is inserted into the femur in the place where the segmental defect occurred. In FIG. 6A, the number 150 indicates the patient's femur that is to be treated. A first femoral implant (100-1) has the unit-cell size of 2 mm and a second femoral implant (100-2) has the unit-cell size of 4 mm. It clearly shows that the first femoral implant (100-1) is formed by the lattice scaffolds which have multiple unit-cells with the size of 2 mm (100-1-1). The second femoral implant (100-2) is formed by the lattice scaffolds which have multiple unit-cells with the size of 4 mm (100-2-1).

In the example shown in FIG. 6A, the target elastic modulus of the femoral bone is identified as 15 GPa (Et=15 GPa). But, the elastic modulus of the implant material (e.g., titanium alloy (Ti6Al4V)) is given as 110 GPa (Eo=110 GPa). Therefore, the adjustment method (FIGS. 1 and 3) of the present invention is applied to create patient specific implants for the target elastic modulus by adjusting lattice scaffolds. As shown in FIG. 6B, both the lattice scaffold designs (100-1 and 100-2) using the BCC (Body-Centered Cubic) unit-cell with 2 mm and 4 mm sizes show the homogenized elastic modulus of 15 GPa (Eh=15 GPa) which is exactly same as the target elastic modulus of 15 GPa. In this example, the densities (ρ) and the strut diameters (D) of the unit-cell are obtained as ρ=0.3662 and D=0.6090 mm for the 100-1 design (2 mm) and ρ=0.4375 and D=1.3620 mm for the 100-2 design (4 mm).

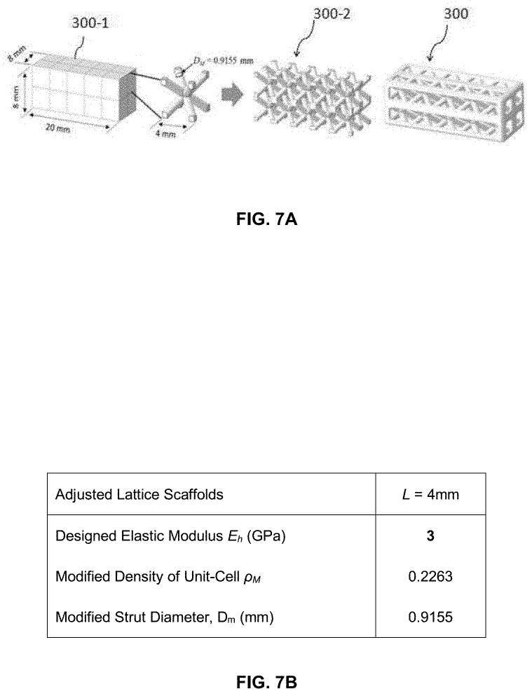

FIG. 7 shows a patient specific implant example of the Lumbar Interbody Fusion for spinal fusion surgery according to the exemplary embodiment of the disclosure. FIG. 7 (a) shows a vertebral implant cage (300) and its internal lattice scaffolds which are formed in a size of 8×8×20 mm by stacking two lattice scaffold structures vertically which include five unit-cells horizontally and two unit-cells laterally (300-1). The Lumbar Interbody Fusion also has the same side effect of stress shielding due to the difference in elastic modulus values between the spinal bone and the implant material. In the vertebral implant cage of this exemplary embodiment (300), a target elastic modulus (Et) of the patient's vertebra area is identified as 3 GPa (Et=3 GPa). The strut diameter and density of the unit-cell are adjusted to match the target elastic modulus since the elastic modulus of the implant material (titanium alloy, Ti6Al4V) is given as 110 GPa (Eo=110 GPa). As shown in FIG. 7 (b), the homogenized elastic modulus (Eh) of the patient specific vertebral implant cage is obtained as 3 GPa which is exactly same as the target elastic modulus (Et). The lattice scaffolds of the obtained implant design through the disclosure have a density (ρ) of 0.2263 and a strut diameter (D) of 0.9155 mm.

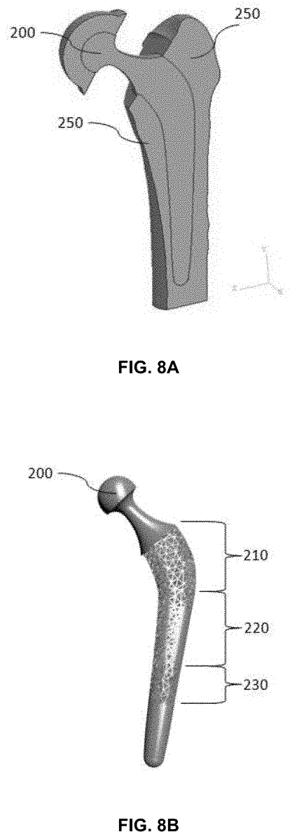

FIG. 8 shows a patient specific femoral stem for a hip implant by using the adjustment method according to the exemplary embodiment of the disclosure. An artificial hip joint implant (200) designed through the disclosure is inserted into a patient's hip joint (250) in the place where the segmental damage occurred. The femoral stem for the patient specific hip implant designed by the disclosure has multiple different elastic modulus values over the domain. For instance, the elastic modulii of three different regions (210, 220, and 230) of the femoral stem in FIG. 8B vary over the domain. Accordingly, each unit-cell of the lattice scaffolds for these regions has different strut diameters and densities to match many different target elastic modulus values (Et) which can be determined from the CT scan images of a patient. The hip joint implant (200) according to the method of the disclosure demonstrates that the elastic modulus values of the entire regions (210, 220, and 230) of the femoral stem are matched to the distribution of the elastic modulus values of the affected bone. Unlike the examples in FIGS. 6 and 7 which use the BCC unit-cell, the hip joint implant utilizes the tetrahedral as the unit-cell structure. Thus, this example shows that the disclosure can be applied with any type of unit-cell structure.

The CAD model of the patient specific hip implant (200) according to the exemplary embodiment of the disclosure is created based on the CT scan images of the patient. Based on the CT scan images, the target elastic modulus values are identified and assigned to the voxel meshes of the femoral stem area. Then, the lattice scaffolds of the femoral stem (200, 210, 220, 230) are designed to have various elastic modulus values ranging from 9 GPa to 124 MPa over the entire domain. FIG. 8B shows that the first region (210) of the femoral stem of the hip joint implant have high bone densities and therefore use large strut diameters and large sizes of unit-cells to achieve corresponding high densities of the lattice scaffolds. The second region (220) and third region (230) of the femoral stem have low bone densities and therefore use relatively narrow strut diameters and small size of unit-cells to achieve low densities of the lattice scaffolds.

FIG. 9 shows the multi-dimensional graphs for the relationship which is identified as a metamodel between the unit-cell size (L), density (ρ), and normalized elastic modulus (Eh/Eo) by using the method according to the exemplary embodiment of the disclosure. The normalized elastic modulus value of the metamodel which is used in the algorithm of FIG. 3 and corresponding contour plot according to the size and density of the unit-cell are shown in FIGS. 9A and 9B. FIG. 9C shows a 2D plot of metamodel according to normalized elastic modulus (Eh/Eo) and density (ρ). The normalized elastic modulus can be calculated as Eh/Eo where Eh is the homogenized elastic modulus for the implant and Eo is the implant material's elastic modulus. By using this normalized elastic modulus, the metamodel can be generalized for any type of desired implant material to be used in the patient specific implant. In other words, by simply changing the Eo value in the normalized elastic modulus (when different material is used for the implant), a new metamodel is not required to be constructed. The as-fabricated contour-plot of the normalized elastic modulus according to the size and density of the unit-cell for a selective laser melting process is shown in FIG. 9D. The differences of the as-fabricated results are clearly visible when compared to FIG. 9B. These differences are caused by the processing parameters of the additive manufacturing technique used for fabrication, which results in deviations between as-designed and as-fabricated unit cells. By using these as-fabricated results, the actual fabricated design parameter values can be estimated to overcome the mismatch issue when the designed lattice scaffold is printed. This process does not require any optimization procedure, and the suitable fabricated values of the design parameters are directly identified from the design space represented with the metamodel.

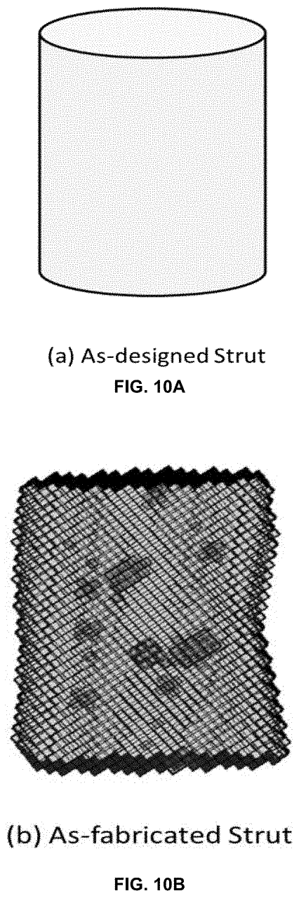

FIG. 10 shows the differences between the as-designed strut and as-fabricated strut. The as-designed strut in FIG. 10A has a constant diameter and homogenous material properties, which are used to estimate the homogenized properties of the unit-cells. However, the homogenized properties determined for the as-designed unit cells will differ when those are fabricated due to manufacturing variations because the as-fabricated struts in FIG. 10B have varying diameter and heterogeneous material with porosity (dark areas in the strut) included, which will influence the resulting homogenized properties of the unit cells. Hence, the metamodel in FIG. 9D is established by accounting for these variations in diameter and material of the struts for the as-fabricated homogenized properties, which can then be used to predict the design parameter values to match the target properties without requiring any optimization process.

The exemplary embodiments of the disclosure are demonstrated with the titanium alloy of TiAl4V6 which has the elastic modulus of 110 GPa. Alternatively, the same process can be applied with different implant materials to match any target elastic modulus of the implant. The disclosure includes the processes of determining the shape of the three-dimensional implant, determining the size of the voxel meshes, selecting the type of the unit-cell, and determining the strut diameters and densities of the unit-cells of the lattice scaffolds to match the target elastic modulus values over the specified domain.

Alternatively, the disclosure includes the following featured operations to adjust a mechanical property (e.g., the elastic modulus) of an implant; 1) specifying a region requiring the implant operation from CT scan images of an affected part, 2) determining an implant shape to be inserted into the specified region, 3) efficiently dividing the implant shape into multiple partitioned three-dimensional regions based on bone density from CT scan images, 4) assigning target elastic modulus (Et) for each of the partitioned three-dimensional regions according to the bone density information obtained from the CT scan images, 5) selecting a specific type of unit-cell to construct the lattice scaffolds with respect to the implant shape, 6) selecting a specific implant material, which has an elastic modulus of Eo, and 7) determining a required strut diameter and density for each unit-cell of the lattice scaffolds in order to minimize the error between the target elastic modulus (Et) and the homogenized elastic modulus (Eh), which is calculated from the implant material's elastic modulus value (Eo) of the selected three-dimensional regions.

In other words, the method of adjusting the elastic modulus of the implant according to the disclosure determines 1) the shape of the implant based on the CT scan images, 2) the target elastic modulus (Et) of the implant based on the elastic modulus extracted from the bone densities of the affected parts, 3) the homogenized elastic modulus (Eh) of the partitioned three-dimensional region based on the implant material's elastic modulus (Eo), and 4) the strut diameter density of each unit-cell of lattice structures to match Eh to Et.

The disclosure creates the partitioned three-dimensional regions from multiple three-dimensional voxel meshes which can be constructed from the CT scan images. In the operation of assigning the target elastic modulus (Et) to each of the segmented three-dimensional regions, the target elastic modulus value can be calculated from the bone density information for each of the three-dimensional voxel meshes according to the CT scan images. In this process, the implant and affected regions are integrally formed into three-dimensional shape using the voxel meshes. Since the relationship between the bone density and its elastic modulus value are highly correlated, the target elastic modulus value of each voxel mesh is determined by extracting the bone density information from the CT scan images. Several analytical formulas are given with reference to Reference documents 9, 10 listed in U.S.provisional application No. 62/626,749.

The disclosure aims to have different target elastic modulus values within the partitioned three-dimensional regions. Accordingly, the strut diameters and densities of each unit-cell of the lattice scaffold are different from each other depending upon the target elastic modulus values of the regions. Therefore, the elastic modulus adjustment method for an implant in the disclosure allows for different dimension, size, and shape of the lattice scaffolds while still allowing these scaffolds to be connected to each other within the partitioned three-dimensional regions.

Along with the elastic modulus adjustment method for the lattice scaffolds, another aspect of the disclosure includes the design process of achieving patient specific implants using the lattice scaffolds, which have adjusted mechanical properties to match the patient's bone properties. The three-dimensional patient-specific implant produced by the disclosure includes an insertion part that is inserted into a specific lesion region, and a non-insertion part that extends from the insertion part of the implant. In order to produce the patient specific implants, the following operations are conducted: 1) dividing the implant into multiple three-dimensional regions, 2) assigning target elastic modulus values (Et) for each of partitioned three-dimensional regions, 3) selecting a specific unit-cell type for the lattice structures with respect to the shape of the implant, and 4) determining the required diameter and density of each strut of the lattice scaffolds to minimize the difference between the target elastic modulus (Et) and the homogenized elastic modulus (Eh), which can be calculated from the implant material's elastic modulus value (Eo) for the three-dimensional regions specified. Once the required strut diameter and density of the unit cell of the lattice scaffolds are determined, the corresponding scaffolds are integrated into the patient specific surgical implants. Then, the final design of the patient specific implant, which has optimal properties to minimize the stress shielding effect, is determined.

The three-dimensional shape of the patient specific implant produced by the disclosure is determined based on the CT scan images of the patient's affected area. In addition, the bone's elastic modulus values of the affected area are extracted from the CT scan images, and these values are used in the design of the implant. The main feature of the patient specific implant produced by the present invention is that elastic modulus values of the implant and the patient's bone are precisely matched over the entire domain of the implant. Accordingly, the implant manufactured through the disclosure can be used longer than the conventional implants by preventing stress shielding effects.

The disclosure includes the following features: three-dimensional regions (as mentioned above) that are created from multiple three-dimensional voxel meshes, three-dimensional voxel meshes that can be constructed based on the CT scan images of the patient, and the operation of assigning the target elastic modulus for each of the three-dimensional voxel meshes includes a procedure of extracting the bone density information from the CT scan images. In addition, the patient specific implant from the disclosure features to have different target elastic modulus values within the partitioned three-dimensional regions. Therefore, it allows the implant to have different strut diameters and densities of the lattice scaffolds while still allowing these scaffolds to be connected to each other within the partitioned three-dimensional regions in the implant.

Although a few embodiments have been described in detail, the present inventive concept is not limited to these embodiments and various changes may be made without departing from the scope defined in the appended claims.

<Description of Symbols>

100: Femoral Implant

100-1: First Femoral Implant

100-1-1: First Size Unit-Cell

100-2 : Second Femoral Implant

100-2-1: Second Size Unit-Cell

150: Insertion Portion of Femoral Implant

200: Hip Implant

210: First Region of Hip Implant

220: Second Region of Hip Implant

230: Third Region of Hip Implant

250: Insertion Portion of Hip Implant

300: Spinal Fusion Implant

300-1: Voxel Mesh of Spinal Fusion Implant

300-2: Lattice Scaffolds of Spinal Fusion Implant

What is claimed is:

1. A method for adjusting mechanical properties of an implant, comprising:
specifying a region that requires an implant operation from a CT scan image of a patient's affected part;
determining an implant shape to be inserted into the specified region;
dividing the implant shape to create a plurality of partitioned three-dimensional regions comprising a plurality of conformal unit cells;
matching a target elastic modulus (Et) for each of the plurality of conformal unit cells with a tensor containing coordinates and density information;
selecting one of multiple types of the conformal unit cell of lattice scaffolds for the implant;
selecting an implant material for the implant; and
adjusting a strut diameter and/or density of the selected type of the lattice scaffolds to minimize a difference between the target elastic modulus (Et) and an as-fabricated homogenized elastic modulus (Eh) for the conformal unit cell, which is calculated from an implant material's elastic modulus value (Eo) obtained from the partitioned three-dimensional regions.

2. The method of claim 1, wherein the plurality of conformal unit cells comprises neighboring regions where the target elastic modulus (Et) are different, resulting in the neighboring regions being connected by unit cells which have different strut diameters and/or densities.

3. The method of claim 1, wherein the size of a plurality of voxel meshes is adjusted while minimizing density information loss to satisfy a practical requirement having a minimum print feature, and while minimizing an effect on matching the target elastic modulus (Et) for the conformal unit cells, by using a modified Structural Similarity Index Method (SSIM) that is developed to handle three-dimensional tensors.

4. The method of claim 1, wherein the as-fabricated homogenized elastic modulus (Eh) for the conformal unit-cell of the lattice scaffolds is estimated by considering manufactured unit cell properties having fabricated diameter values using a multiscale modeling method with the elastic modulus of the implant material (Eo).

5. The method of claim 1, wherein fabricated values of design parameters of the conformal unit-cells of the lattice scaffolds are determined by using a metamodel that utilizes a unit-cell size (L), a normalized elastic modulus (Eh/Eo), and a unit-cell density (ρ) without requiring any optimization procedure.

6. The method of claim 1, wherein a type of the conformal unit-cells of the lattice scaffolds is selected at least one among crossed, cantley, octet, Paramount1, Diagonal, Paramount2, Midpoint, or tetrahedral or body centered cubic (BCC) unit cell.
